(19) United States
(12) Patent Application Publication
(10) Pub. No.: US 2008/0220074 A1
(43) Pub. Date: Sep. 11, 2008

(54) GAMMA RADIATION STERILIZED NANOPARTICULATE DOCETAXEL COMPOSITIONS AND METHODS OF MAKING SAME

(75) Inventors: H. William Bosch, Bryn Mawr, PA (US); Janine Keller, Collegeville, PA (US); Niels Ryde, Malvern, PA (US)

Correspondence Address: Fox Rothschild, LLP;Elan Pharma International Limited, 2000 Market Street, Philadelphia, PA, 19103, US

(73) Assignee: ELAN CORPORATION PLC, Dublin (IE)

(21) Appl. No.: 12/052,436

(22) Filed: Mar. 20, 2008

Related U.S. Application Data
(63) Continuation-in-part of application No. 10/654,600, filed on Sep. 4, 2003.
(60) Provisional application No. 60/415,749, filed on Oct. 4, 2002.
(60) Provisional application No. 60/896,647, filed on Mar. 23, 2007.

(51) Int. Cl. A61K 9/14; A61K 31/337

(52) U.S. Cl. 424/489; 514/449

(57) ABSTRACT

Nanoparticulate compositions comprising docetaxel or a salt, derivative, conjugate or analogue thereof, wherein the compositions are terminally sterilized via gamma radiation, are described, as well as methods of making and using such compositions.

### CROSS-REFERENCE TO RELATED APPLICATIONS

This application (1) is a continuation-in-part of U.S. patent application Ser. No. 10/654,600, filed on Sep. 4, 2003, which claims benefit of U.S. Provisional Patent Application No. 60/415,749, filed on Oct. 4, 2002; and (2) claims benefit of U.S. Provisional Patent Application No. 60/896,647, filed on Mar. 23, 2007. Each of these applications is incorporated herein by reference in its entirety.

### FIELD OF THE INVENTION

The present invention relates to nanoparticulate compositions of docetaxel, and in particular, a terminally sterilized nanoparticulate composition useful in the treatment of cancer, particularly, breast, ovarian, prostate, and lung cancer.

### BACKGROUND OF THE INVENTION

Taxoids or taxanes are compounds that inhibit cell growth by stopping cell division, and include docetaxel and paclitaxel. They are also called antimitotic or antimicrotubule agents or mitotic inhibitors.

Taxoid-based compositions having anti-tumor and anti-leukemia activity, and the use thereof, are described in U.S. Pat. No. 5,438,072. U.S. Pat. No. 6,624,317 refers to the preparation of taxoid conjugates for use in the treatment of cancer. FIG. 1A of U.S. Pat. No. 5,508,447 to Magnus (the “Magnus patent”) shows the structure and numbering of the taxane ring system. The Magnus patent is directed to the synthesis of taxol for use in cancer treatment. U.S. Pat. Nos. 5,698,582 and 5,714,512 relate to taxane derivatives used in pharmaceutical compositions suitable for injection as anti-tumor and anti-leukemia treatments. U.S. Pat. Nos. 6,028,206 and 5,614,645 relate to the preparation of taxol analogues that are useful in the treatment of cancer. U.S. Pat. Nos. 4,814,470 and 5,411,984 both relate to the preparation of certain taxol derivatives for use in the treatment of cancer. All of the aforementioned patents are incorporated by reference herein.

Docetaxel is a semi-synthetic, antineoplastic agent belonging to the taxoid family. Docetaxel is a white to almost-white powder; it is highly lipophilic and practically insoluble in water. The chemical name for docetaxel is (2R,3S)—N-carboxy-3-phenylisoserine, N-tert-butyl ester, 13-ester with 5β-20-epoxy-1,2α,4,7β, 10β, 13α-hexahydroxytax-11-en-9-one 4-acetate 2-benzoate. One method for preparing docetaxel is by semisynthesis beginning with a precursor (taxoid 10-deacetylbaccatin III) extracted from the renewable needle biomass of yew plants.

Docetaxel may be formulated into nanoparticulates as described in co-pending, and commonly owned, U.S. patent application Ser. No. 11/361,055. Nanoparticulate active agent compositions in general, are described in U.S. Pat. No. 5,145,684 (“the '684 patent”), the contents of which are incorporated by reference herein. The '684 patent teaches nanoparticles of a poorly soluble therapeutic or diagnostic agent having adsorbed onto or associated with the surface thereof a non-crosslinked surface stabilizer.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles,” each of which is incorporated herein by reference.

It is desirable that pharmaceutical products, once manufactured, have a sufficient shelf life such that the product can be stored at room temperature at an end user location before administration. It is generally known that solid formulations of a pharmaceutical product are more stable than a liquid formulation of the same pharmaceutical product. One method of converting a liquid formulation into a semi-solid formulation is through the process of lyophilization.

A lyophilization formulation typically contains three general components, the active ingredient, excipients, and the solvent. Excipients serve several functions, but primarily provide a stable environment for the active ingredient. The excipients may cryoprotect the active ingredient during the freezing process and/or may serve as bulking agents that enhance the structural quality of the lyo cake.

In addition to having a sufficient shelf life, pharmaceutical products should also be sterile before use. Commonly used methods for sterilizing pharmaceutical products after manufacture and before end use include: heat sterilization, sterile filtration, and radiation. Not all of these sterilization methods are useful for sterilizing nanoparticulate compositions, and each method has its drawback.

### 1. Heat Sterilization of Nanoparticulate Active Agent Compositions

One of the problems that may be encountered with heat sterilization of nanoparticulate active agent compositions is the solubilization and subsequent recrystallization of the component active agent particles. This process results in an increase in the size distribution of the active agent particles. In cases where the nanoparticulate active agent formulations contain surface modifiers, which have cloud points lower than the sterilization temperature (generally about 121° C.), it is theorized that the structure of the surface modifiers collapses which results in the nanoparticulate active agent precipitating from solution at or below the sterilization temperature. Thus, some nanoparticulate active agent formulations also exhibit particle aggregation following exposure to elevated temperatures during the heat sterilization process.

Crystal growth and particle aggregation in nanoparticulate active agent preparations are highly undesirable. The presence of large crystals in the nanoparticulate active agent composition may cause undesirable side effects, especially when the preparation is in an injectable formulation. Larger particles formed by particle aggregation and recrystallization can interfere with blood flow, causing pulmonary embolism and death.

### 2. Sterile Filtration

Filtration is an effective method for sterilizing homogeneous solutions when the membrane filter pore size is less than or equal to about 0.2 microns (200 nm) because a 0.2 micron filter is sufficient to remove essentially all bacteria. Sterile filtration is typically not used to sterilize conventional suspensions of micron-sized drug particles because the drug substance particles are too large to pass through the membrane pores. Sterile filtration is also not typically used to sterilize nanoparticulate formulation because although a nanoparticulate composition may have a mean particle size less than 0.2 μm, there is a portion of the population of the particles that makes up the mean that is larger than 0.2 microns. Thus, when passed through a 0.2 μm filter, typical nanoparticulate compositions suffer the same fate as micron-sized compositions: they clog the sterilizing filter. Thus, only nanoparticulate active agent compositions having a very small average particle size where the larger-sized particles contributing to the mean particle size are not larger than 0.2 μm can be sterile filtered.

### 3. Gamma Radiation

Gamma radiation is a common and valid method to sterilize pharmaceutical products. However, one disadvantage to gamma radiation is that, prior to it use, the effect that the radiation will have on the components of a pharmaceutical formulation must be determined. For example, U.S. Pat. No. 5,362,442 reports that gamma radiation of certain sugars in solution, particularly glucose, has been reported to decompose the sugars in the solutions. Because each component of the formulation (e.g., each individual excipient in a nanoparticulate composition) reacts differently to ionizing radiation, one must verify that the maximum dose likely to be administered during the sterilization process will not adversely affect the quality, safety or performance of the nanoparticulate composition throughout its shelf life.

There is currently a need for terminally sterilized, docetaxel formulations that have enhanced solubility characteristics which, in turn, provide enhanced bioavailability and reduced toxicity upon administration to a patient, wherein the formulation has been sterilized by gamma radiation. The present invention satisfies these needs by providing sterilized compositions comprising nanoparticulate formulations of docetaxel and analogues thereof, as well as methods for making the same. Such formulations include, but are not limited to, redispersible lyos of injectable nanoparticulate docetaxel or analogues thereof.

### SUMMARY OF THE INVENTION

In certain aspects, the present invention relates to solid nanoparticulate compositions comprising docetaxel or an analogue thereof, wherein the compositions are terminally sterilized via gamma radiation, as well as methods of making and using the same.

In one aspect of the invention, the composition comprises particles comprising docetaxel or an analogue thereof, wherein the particles have an average size of less than about 2000 nm. The composition may also comprise at least one surface stabilizer adsorbed onto or associated with the surface of the particles. The composition is sterilized by exposure to gamma radiation.

Further aspects of the present invention are directed to methods of making compositions according to the invention.

Additional aspects of the present invention are directed to methods of treating a subject with a gamma radiated solid nanoparticulate docetaxel dosage form comprising administering to the subject an effective amount of a gamma radiated nanoparticulate dosage composition comprising docetaxel or an analogue thereof.

Both the foregoing general description and the following detailed description are exemplary and explanatory, and are intended to provide further explanation of the invention as claimed. Other objects, advantages, and novel features will be readily apparent to those skilled in the art from the following detailed description of the invention.

### DETAILED DESCRIPTION OF THE INVENTION

### Definitions

As employed above and throughout the disclosure, the following terms, unless otherwise indicated, shall be understood to have the following meanings.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, a “stable” docetaxel or analogue thereof particle connotes, but is not limited to a docetaxel or analogue thereof with one or more of the following parameters: (1) the docetaxel or analogue thereof particles do not appreciably flocculate or agglomerate due to interparticle attractive forces or otherwise significantly increase in particle size over time; (2) the physical structure of the docetaxel or analogue thereof particles is not altered over time, such as by conversion from an amorphous phase to a crystalline phase; (3) the docetaxel or analogue thereof particles are chemically stable; and/or (4) where the docetaxel or analogue thereof has not been subject to a heating step at or above the melting point of the docetaxel or analogue thereof in the preparation of the nanoparticles of the invention.

The term “conventional” or “non-nanoparticulate” active agent or docetaxel or analogue thereof shall mean an active agent, such as docetaxel or analogue thereof, which is solubilized or which has an effective average particle size of greater than about 2000 nm. Nanoparticulate active agents as defined herein have an effective average particle size of less than about 2000 nm.

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

As used herein, the phrase “therapeutically effective amount” means the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “microbial” with respect to contamination, as used herein is deemed to include all biological contaminants including bacteria, yeast, and molds.

The terms “sterilize” or “sterilized” as used in the present application generally means to inactivate biological contaminants present in the product. In typical pharmaceutical applications, exposure to at least a 25 kGray dose of radiation sterilizes the pharmaceutical product. Suitable exemplary sterilization by radiation techniques, among other sterilization techniques, are described in USP<1212>(USP29-NF24)_, Sterilization and Sterility Assurance of Compendial Articles.

In certain aspects, the present invention is directed to the surprising discovery that solid forms of nanoparticulate compositions comprising docetaxel or an analogue as an active agent can be successfully terminally sterilized via gamma radiation. The solid that is sterilized according to aspects of this invention can be formulated into any suitable dosage form. Embodiments of the present invention include liquid compositions comprising reconstituted solid nanoparticulate compositions comprising docetaxel or an analogue that are sterilized via gamma radiation.

In one aspect of the invention, the nanoparticulate compositions are comprised of particles containing a pharmaceutically active ingredient, which may be docetaxel, a salt, derivative, conjugate or analogue thereof. Preferably, the particles have an effective average particle size of less than about 2000 nm. The compositions may also comprise at least one surface stabilizer adsorbed onto or associated with the surface of the particles. The compositions are sterilized by exposure to gamma radiation. In certain aspects of the invention, after gamma radiation and reconstitution in a liquid media, the sterilized solid redisperses into a particle size which is substantially similar to the original nanoparticulate particle size prior to incorporation into a solid.

Additional aspects of the invention are directed to methods of making compositions according to the invention. According to one aspect of the invention, a method for making a sterilized nanoparticulate docetaxel composition comprises the steps of mixing docetaxel, optionally in the presence of at least one excipient, and at least one surface stabilizer in an aqueous medium containing milling media for a period of time and under conditions sufficient to provide a dispersion of particles of docetaxel having an effective average particle size of less than about 2000 nm and such that the at least one surface stabilizer is adsorbed on the surface of the particles; removing the milling media from the dispersion; lyophilizing the dispersion to form a lyo; and sterilizing the lyo to produce a sterilized docetaxel composition.

Another aspect of the invention encompasses a method of treating a subject in need comprising administering a therapeutically effective amount of a solid sterilized nanoparticulate composition comprising docetaxel or an analogue according to the invention. Another aspect of the invention is a method of treating a mammal in need comprising administering a therapeutically effective amount of a liquid composition comprising a reconstituted solid nanoparticulate composition comprising docetaxel or an analogue sterilized via gamma radiation.

### Docetaxel

As used herein, the term “docetaxel” includes analogues, derivatives, conjugates, and salts thereof, and can be in a crystalline phase, an amorphous phase, a semi-crystalline phase, a semi-amorphous phase, or a mixture thereof. Docetaxel or an analogue thereof may be present either in the form of one substantially optically pure enantiomer or as a mixture, racemic or otherwise, of enantiomers.

Analogues of docetaxel described and encompassed by the invention include, but are not limited to,

(1) docetaxel analogues comprising cyclohexyl groups instead of phenyl groups at the C-3′ and/or C-2 benzoate positions, such as 3′-dephenyl-3′cyclohexyldocetaxel, 2-(hexahydro)docetaxel, and 3′-dephenyl-3′cyclohexyl-2-(hexahydro)docetaxel (Ojima et al., “Synthesis and structure-activity relationships of new antitumor taxoids. Effects of cyclohexyl substitution at the C-3′ and/or C-2 of taxotere (docetaxel),” J. Med. Chem., 37(16):2602-8 (1994));

(2) docetaxel analogues lacking phenyl or an aromatic group at C-3′ or C-2 position, such as 3′-dephenyl-3′-cyclohexyldocetaxel and 2-(hexahydro)docetaxel;

(3) 2-amido docetaxel analogues, including m-methoxy and m-chlorobenzoylamido analogues (Fang et al., Bioorg. Med. Chem. Lett., 12(11):1543-6 (2002);

(4) docetaxel analogues lacking the oxetane D-ring but possessing the 4alpha-acetoxy group, which is important for biological activity, such as 5(20)-thia docetaxel analogues, which can be synthesized from 10-deacetylbaccatin III or taxine B and isotaxine B, described in Merckle et al., “Semisynthesis of D-ring modified taxoids: novel thia derivatives of docetaxel,” J. Org. Chem., 66(15):5058-65 (2001), and Deka et al., Org. Lett., 5(26):5031-4 (2003);

(5) 5(20)deoxydocetaxel;

(6) 10-deoxy-10-C-morpholinoethyl docetaxel analogues, including docetaxel analogues in which the 7-hydroxyl group is modified to hydrophobic groups (methoxy, deoxy, 6,7-olefin, alpha-F, 7-beta-8-beta-methano, fluoromethoxy), described in Iimura et al., “Orally active docetaxel analogue: synthesis of 10-deoxy-10-C-morpholinoethyl docetaxel analogues,” Bioorg. Med. Chem. Lett., 11(3):407-10 (2001);

(7) docetaxel analogues described in Cassidy et al., Clin. Can. Res., 8:846-855 (2002), such as analogues having a t-butyl carbamate as the isoserine N-acyl substituent, but differing from docetaxel at C-10 (acetyl group versus hydroxyl) and at the C-13 isoserine linkage (enol ester versus ester);

(8) docetaxel analogues having a peptide side chain at C3, described in Larroque et al., “Novel C2-C3” N-peptide linked macrocyclic taxoids. Part 1: Synthesis and biological activities of docetaxel analogues with a peptide side chain at C3”, Bioorg. Med. Chem. Lett. 15(21):4722-4726 (2005);

(9) XRP9881 (10-deacetyl baccatin III docetaxel analogue);

(10) XRP6528 (10-deacetyl baccatin III docetaxel analogue);

(11) Ortataxel (14-beta-hydroxy-deacetyl baccatin III docetaxel analogue);

(12) MAC-321 (10-deacetyl-7-propanoyl baccatin docetaxel analogue);

(13) DJ-927 (7-deoxy-9-beta-dihydro-9,10, 0-acetal taxane docetaxel analogue);

(14) docetaxel analogues having C2-C3′N-linkages bearing an aromatic ring at position C2, and tethered between N3′ and the C2-aromatic ring at the ortho, meta, or para position. The para-substituted derivatives were unable to stabilize microtubules, whereas the ortho- and meta-substituted compounds show significant activity in cold-induced microtubule disassembly assay. Olivier et al., “Synthesis of C2-C3′N-Linked Macrocyclic Taxoids; Novel Docetaxel Analogues with High Tubulin Activity,” J. Med. Chem., 47(24:5937-44 (November 2004);

(15) docetaxel analogues bearing 22-membered (or more) rings connecting the C-2 OH and C-3′ NH moieties (biological evaluation of docetaxel analogues bearing 18-, 20-, 21-, and 22-membered rings connecting the C-2 OH and C-3′ NH moieties showed that activity is dependent on the ring size; only the 22-membered ring taxoid 3d exhibited significant tubulin binding) (Querolle et al., “Synthesis of novel macrocyclic docetaxel analogues. Influence of their macrocyclic ring size on tubulin activity,” J. Med. Chem., 46(17):3623-30 (2003).);

(16) 7beta-O-glycosylated docetaxel analogue (Anastasia et al., “Semi-Synthesis of an O-glycosylated docetaxel analogue,” Bioorg. Med. Chem., 11(7):1551-6 (2003));

(17) 10-alkylated docetaxel analogues, such as a 10-alkylated docetaxel analogue having a methoxycarbonyl group at the end of the alkyl moiety (Nakayama et al., “Synthesis and cytotoxic activity of novel 10-alkylated docetaxel analogs,” Bioorg. Med. Chem. Lett., 8(5):427-32 (1998));

(18) 2′,2′-difluoro, 3′-(2-furyl), and 3′-(2-pyrrolyl) docetaxel analogues (Uoto et al., “Synthesis and structure-activity relationships of novel 2′,2′-difluoro analogues of docetaxel,” Chem. Pharm. Bull. (Tokyo), 45(11):1793-804 (1997)); and

(19) Fluorescent and biotinylated docetaxel analogues, such as docetaxel analogues that possess (a) a N-(7-nitrobenz-2-oxa-1,3-diazo-4-yl)amido-6-caproyl chain in position 7 or 3′, (b) a N-(7-nitrobenz-2-oxa-1,3-diazo-4-yl)amido-3-propanoyl group at 3′, or (c) a 5′-biotinyl amido-6-caproyl chain in position 7, 10 or 3′ (Dubois et al., “Fluorescent and biotinylated analogues of docetaxel: synthesis and biological evaluation,” Bioorg. Med. Chem., 3(10):1357-68 (1995)).

### Compositions

According to certain aspects of the invention, the composition is formulated for administration via any pharmaceutically acceptable route of administration, including, but not limited to, oral, pulmonary, rectal, opthalmic, colonic, parenteral, intracisternal, intravaginal, intraperitoneal, local, buccal, nasal, and topical administration.

In certain aspects of the invention, the composition is formulated into any pharmaceutically acceptable dosage form, including, but not limited to, liquid dispersions, solid dispersions, liquid-filled capsule, gels, aerosols, ointments, creams, lyophilized formulations, tablets, capsules, multi-particulate filled capsule, tablet composed of multi-particulates, compressed tablet, and a capsule filled with enteric-coated beads of the active ingredient.

According to certain embodiments of the invention, the inclusion of one or more sugars is useful in preparing the compositions. Without intending to be bound by any theory or theories of operation, it is believed that sugars may serve one or more functions. For example, sugars may act as surface modifiers, as crystal growth inhibitors, as bulking agents and/or may act to prevent aggregation of particles. Examples of sugars useful in compositions of the invention include, but are not limited to, sucrose, mannitol, dextrose, lactose, sorbitol, maltose, trehalose, and other sugars.

According to one embodiment of the invention, a lyophilized dosage form is exposed to a sufficient amount of radiation to sterilize the dosage form. Exemplary amounts of gamma radiation include, but are not limited to, amounts of gamma radiation providing a total dose of radiation from about 5 to about 50 kGray, about 15 kGray to about 40 kGray, about 15 to about 30 kGray, about 20 to about 30, or about 25 to about 40 kGray. In one embodiment of the invention, sterilization is accomplished by exposing the lyo to about 25 kGray of gamma radiation.

In a preferred embodiment, the composition is formulated for use in an injectable dosage form.

In another aspect of the invention, the composition is formulated into dosage forms including, but not limited to, controlled release formulations, fast melt formulations, delayed release formulations, extended release formulations, pulsatile release formulations, and mixed immediate release and controlled release formulations.

The invention provides compositions comprising nanoparticulate docetaxel or analogue thereof particles and at least one surface stabilizer. The surface stabilizers are preferably adsorbed onto or associated with the surface of the docetaxel or analogue thereof particles. Surface stabilizers useful herein do not chemically react with the docetaxel or analogue thereof particles or itself. In another embodiment, the compositions of the present invention can comprise two or more surface stabilizers.

Surface stabilizers useful herein physically adhere on or associate with the surface of the nanoparticulate active agent but do not chemically react with the active agent particles.

Exemplary useful surface stabilizers include, but are not limited to, known organic and inorganic pharmaceutical excipients, as well as peptides and proteins. Such excipients include various polymers, low molecular weight oligomers, natural products, and surfactants. Useful surface stabilizers include nonionic surface stabilizers, anionic surface stabilizers, cationic surface stabilizers, and zwitterionic surface stabilizers. Combinations of more than one surface stabilizer can be used in the invention.

Representative examples of surface stabilizers include, but are not limited to, hydroxypropyl methylcellulose, hydroxypropylcellulose, polyvinylpyrrolidone (PVP), random copolymers of vinyl pyrrolidone and vinyl acetate, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxes 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.); Tetronic 1508® (T-1508) (BASF Wyandotte Corporation), Tritons X-200®, which is an alkyl aryl polyether sulfonate (Dow); Crodestas F-110®, which is a mixture of sucrose stearate and sucrose distearate (Croda Inc.); p-isononylphenoxypoly-(glyc-idol), also known as Olin-10G® or Surfactant 10-G® (Olin Chemicals, Stamford, Conn.); Crodestas SL-40.RTM. (Croda, Inc.); and SA9OHCO, which is C18H37CH2C(O)N(CH3)-CH2(CHOH)4(CH20H)2 (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, and the like.

Additional examples of useful surface stabilizers include, but are not limited to, polymers, biopolymers, polysaccharides, cellulosics, alginates, phospholipids, poly-n-methylpyridinium chloride, anthryul pyridinium chloride, cationic phospholipids, chitosan, polylysine, polyvinylimidazole, polybrene, polymethylmethacrylate trimethylammonium bromide (PMMTMABr), hexyldecyltrimethylammonium bromide (HDMAB), and polyvinylpyrrolidone-2-dimethylaminoethyl methacrylate dimethyl sulfate.

Other useful stabilizers include, but are not limited to, cationic lipids, sulfonium, phosphonium, and quarternary ammonium compounds, stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl(ethenoxy)4 ammonium chloride or bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Other useful cationic surface stabilizers are described in J. Cross and E. Singer, Cationic Surfactants Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Examples of preferred surface stabilizers useful in certain embodiments of the present invention include, but are not limited to, poloxamer 188, poloxamer 338, poloxamer 407, polysorbate 80, and lecithin.

Other known pharmaceutical excipients and surface stabilizers and are described in detail in the Handbook of Pharmaceutical Excipients, published jointly by the American Pharmaceutical Association and The Pharmaceutical Society of Great Britain (The Pharmaceutical Press, 2000), specifically incorporated herein by reference. Pharmaceutical excipients listed therein include, acacia, acesulfame potassium, albumin, alcohol, alginic acid, aliphatic polyesters, alpha tocopherol, ascorbic acid, ascorbyl palmitate, aspartame, bentonite, benzalkonium chloride, benzethonium chloride, benzoic acid, benzyl alcohol, benzyl benzoate, bronopol, butylated hydroxyanisole, butylated hdroxytoluene, butylparaben, calcium carbonate, calcium phosphate dibasic anhydrous, calcium phosphate dibasic dehydrate, calcium phosphate tribasic, calcium stearate, calcium sulfate, canola oil, carbomer, carbon dioxide, carboxymethylcellulose calcium, carboxymethylcellulose sodium, carrageenan, castor oil, hydrogenated cellulose acetate, cellulose acetate phthalate, powdered microcrystalline cellulose, silicified microcrystalline cellulose, cetostearyl alcohol, cetrimide, cetyl alcohol, chlorhexidine, chlorobutanol, chlorocresol, chlorodifluoroethane (HCFC), chlorofluorocarbons (cFC), cholesterol, citric acid monohydrate, colloidal silicon dioxide, coloring agents, corn oil, cottonseed oil, cresol, croscarmellose sodium, crospovidone, cyclodextrins, dextrates, dextrin, dextrose, dibutyl sebacate, diethanolamine, diethyl phthalate, difluoroethane (HFC), dimethyl ether, docusate sodium, edetic acid, ethylcellulose, ethyl maltol, ethyl oleate, ethylparaben, ethyl vanillin, fructose, fumaric acid, gelatin, glucose, liquid glycerin, glyceryl monooleate, glyceryl monostearate, glyceryl palmitostearate, glycofurol, guar gum, heptafluoropropane (HFC), hydrocarbons (HC), hydrochloric acid, hydroxyethyl cellulose, hydroxypropyl cellulose, low-substituted hydroxypropyl cellulose, hydroxypropyl methylcellulose, hydroxypropyl methylcellulose phthalate, imidurea, isopropyl alcohol, isopropyl myristate, isopropyl palmitate, kaolin, lactic acid, lactitol, lactose, lanolin, lanolin alcohols, hydrous lanolin, lecithin, magnesium aluminum silicate, magnesium carbonate, magnesium oxide, magnesium stearate, magnesium trisilicate, malic acid, maltitol, maltitol solution, maltodextrin, maltol, maltose, mannitol, medium chain triglycerides, meglumine, menthol, methylcellulose, methylparaben, mineral oil, light mineral oil, mineral oil and lanolin alcohols, monoethanolamine, nitrogen, nitrous oxide, oleic acid, paraffin, peanut oil, petrolatum, petrolatum and lanolin alcohols, phenol, phenoxyethanol, phenylethyl alcohol, phenylmercuric acetate, phenylmercuric borate, phenylmercuric nitrate, polacrilin potassium, poloxamer, polydextrose, polyethylene glycol, polyethylene oxide, polymethacrylates, polyoxyethylene alkyl ethers, polyoxyethylene castor oil derivatives, polyoxyethylene sorbitan fatty acid esters, polyoxyethylene stearates, polyvinyl alcohol, potassium chloride, potassium citrate, potassium sorbate, povidone, propylene carbonate, propylene glycol, propylene glycol alginate, propyl gallate, propylparaben, saccharin, saccharin sodium, sesame oil, shellac, sodium alginate, sodium acorbate, sodium benzoate, sodium bicarbonate, sodium chloride, sodium citrate dehydrate, sodium cyclamate, sodium lauryl sulfate, sodium metabisulfite, dibasic sodium phosphate, monobasic sodium phosphate, sodium propionate, sodium starch glycolate, sodium stearyl fumarate, sorbic acid, sorbitan esters (sorbitan fatty acid esters), sorbitol, soybean oil, starch, starch, pregelatinized starch, sterilizable maize, stearic acid, stearyl alcohol, sucrose, compressible sugar, confectioner's sugar, sugar spheres, suppository bases, hard fat, talc, tartaric acid, tetrafluoroethane (HFC), thimerosal, titanium dioxide, tragacanth, triacetin, triethanolamine, triethyl citrate, vanillin, type I hydrogenated vegetable oil, water, anionic emulsifying wax, Carnauba wax, cetyl esters wax, microcrystalline wax, nonionic emulsifying wax, white wax, yellow wax, xanthan gum, xylitol, zein, and zinc stearate.

In certain other embodiments of the invention, the composition may comprise at least one peptide or protein as a surface stabilizer adsorbed onto, or associated with, the surface of the active agent. The peptide and/or protein surface stabilizer can be contacted with the active agent either before, preferably during, or after size reduction of the active agent.

### Concentration of Nanoparticulate Docetaxel and Surface Stabilizers

The relative amounts of docetaxel or analogue thereof and one or more surface stabilizers can vary widely. The optimal amount of the individual components depends, for example, upon physical and chemical attributes of the surface stabilizer(s) and docetaxel or analogue thereof selected, such as the hydrophilic lipophilic balance (HLB), melting point, and the surface tension of water solutions of the stabilizer, etc.

The concentrations of the components of the present invention are measured by % w/w of the dry composition. As would be understood by one of ordinary skill in the art, the amounts of the components in the dry composition can be converted to account for the aqueous dispersion medium when the composition is in a liquid dispersion form.

Preferably, the concentration of the docetaxel or analogue thereof in a dry, lyophilized composition can be present in about 1%, 5%, 10%, 20%, 30%, 40%, 50%, 60%, 70%, 80%, 90%, 95% or 99%, by weight, based on the total combined weight of the docetaxel or analogue thereof and at the least one surface stabilizer, not including other excipients.

Preferably, the concentration of at least one surface stabilizer in the dry lyophilized composition can be about 1%, 5%, 10%, 20,%, 30%, 40%, 50%, 60%, 70%, 80%, 90%, 95%, or 99%, by weight, based on the total combined dry weight of the docetaxel or analogue thereof and the at least one surface stabilizer, not including other excipients.

### Other Pharmaceutical Excipients

The present invention also includes nanoparticulate docetaxel or analogue thereof compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like. In certain embodiments of the invention, the nanoparticulate docetaxel or analogue thereof formulations are in an injectable form.

Non-limiting examples of excipients that may be included in the dry composition are bulking agents, crystal growth inhibitors, free radical scavenger agents, and redispersion agents. Preferably, the excipients may be present in an amount from about 5 to about 95, about 10 to about 95, about 20 to about 95, about 50 to about 90, about 60 to about 90, about 70 to about 90, or about 70 to about 80, as measured by % w/w of the dry composition.

In one embodiment of the invention, the excipients are preferably present in an amount from about 5 to about 95, about 10 to about 95, about 20 to about 95, about 50 to about 90, about 60 to about 90, about 70 to about 90, or about 70 to about 80, measured by % w/w of the dry composition.

Pharmaceutical compositions according to aspects of the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Compositions suitable for parenteral injection may comprise, for example, physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents, or vehicles including water, ethanol, sodium chloride, Ringer's solution, lactated Ringer's solution, stabilizer solutions, tonicity enhancers (sucrose, dextrose, mannitol, etc.) polyols (propyleneglycol, polyethylene-glycol, glycerol, and the like), suitable mixtures thereof, vegetable oils (such as olive oil) and injectable organic esters such as ethyl oleate. Suitable fluids are referenced in Remington's Pharmaceutical Sciences, 17th edition, published by Mack Publishing Co., page 1543.

### Injectable Nanoparticulate Docetaxel Formulations

In one embodiment of the invention, provided are injectable nanoparticulate docetaxel or analogue thereof formulations that can comprise high concentrations in low injection volumes, with rapid dissolution upon administration.

Exemplary preservatives useful in certain embodiments of the invention include, without limitation, methylparaben (about 0.18% based on % w/w), propylparaben (about 0.02% based on % w/w), phenol (about 0.5% based on % w/w), and benzyl alcohol (up to 2% v/v). An exemplary pH adjusting agent is sodium hydroxide, and an exemplary liquid carrier is sterile water for injection. Other useful preservatives, pH adjusting agents, and liquid carriers are well-known in the art.

### Nanoparticulate Docetaxel Particle Size

Particle size may be measured by any conventional particle size measuring techniques well known to those skilled in the art. Such techniques include, for example, sedimentation field flow fractionation, photon correlation spectroscopy, light scattering, and disk centrifugation. An exemplary machine utilizing light scattering measuring techniques is the Horiba LA-910 Laser Scattering Particle Size Distribution Analyzer manufactured by Horiba, Ltd. of Minami-ku Kyoto, Japan.

The above-mentioned measuring techniques typically report the particle size of a composition as a statistical distribution. Accordingly, from this distribution, one of ordinary skill in the art can calculate a mean, median, and mode, as well as visually depict the distribution as a probability density function. Furthermore, percentile ranks of the distribution can be identified.

As would be understood by one of ordinary skill in the art, the distribution can be defined on the basis of a number distribution, a weight distribution, or volume distribution of solid particles. Preferably, the particle size distributions of the present invention are defined according to a weight distribution.

As used herein, “effective average particle size” means that for a given particle size, x, 50% of the particle population are a size, by weight, of less than x, and 50% of the particle population are a size, by weight, that is greater than x. For example, a composition comprising particles of docetaxel, derivatives of docetaxel, conjugates of docetaxel and analogues of docetaxel, that have an “effective average particle size of 2000 nm” means that 50% of the particles are of a size, by weight, smaller than about 2000 nm and 50% of the particles are of a size, by weight, that is larger than 2000 nm.

Compositions of the invention comprise docetaxel or an analogue thereof particles having an effective average particle size of less than about 2 microns. In other embodiments of the invention, the docetaxel or analogue thereof particles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

In another embodiment of the invention, the compositions of the invention are in an injectable dosage form and the docetaxel or analogue thereof particles preferably have an effective average particle size of less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm. Injectable compositions can comprise docetaxel or an analogue thereof having an effective average particle size of greater than about 1 micron, up to about 2 microns.

As used herein, the nomenclature “D” followed by a number, e.g., D50, is the particle size at which 50% of the population of particles are smaller and 50% of the population of particles are larger. In another example, the D90 of a particle size distribution is the particle size below which 90% of particles fall, by weight; and which conversely, only 10% of the particles are of a larger particle size, by weight.

As used herein, the term “Dmean” is the numerical average for the population of particles in a composition. For example, if a composition comprises 100 particles, the total weight of the composition is divided by the number of particles in the composition.

The gamma radiation-sterilized solid nanoparticulate compositions of the invention preferably redisperse upon reconstitution in suitable vehicles such that the effective average particle size of the redispersed active agent particles is less than about 2 microns. This is significant, because upon administration the nanoparticulate active agent compositions of the invention did not redisperse to a substantially nanoparticulate particle size, then the dosage form may lose the benefits afforded by formulating the active agent into a nanoparticulate particle size.

This is because nanoparticulate active agent compositions benefit from the small particle size of the active agent; if the active agent does not redisperse into the small particle sizes upon administration, then “clumps” or agglomerated active agent particles are formed, owing to the extremely high surface free energy of the nanoparticulate active agent system and the thermodynamic driving force to achieve an overall reduction in free energy. With the formation of such agglomerated particles, parenteral administration of the particles could lead to serious toxicity resulting from emboli or capillary occlusion. Furthermore, the bioavailability of the dosage form may fall well below that observed with a form of the nanoparticulate active agent that does not form such agglomerated particles.

In other embodiments of the invention, the redispersed particles of the invention (redispersed in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

### Methods of Making Nanoparticulate Active Agent Compositions

According to certain aspects of the invention, nanoparticulate active agent compositions can be made using methods known in the art such as, for example, milling, homogenization, and precipitation techniques. Exemplary methods of making nanoparticulate active agent compositions are described in U.S. Pat. No. 5,145,684.

Methods of making nanoparticulate active agent compositions are also described in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” U.S. Pat. No. 5,665,331, for “Co-Microprecipitation of Nanoparticulate Pharmaceutical Agents with Crystal Growth Modifiers;” U.S. Pat. No. 5,662,883, for “Co-Microprecipitation of Nanoparticulate Pharmaceutical Agents with Crystal Growth Modifiers;” U.S. Pat. No. 5,560,932, for “Microprecipitation of Nanoparticulate Pharmaceutical Agents;” U.S. Pat. No. 5,543,133, for “Process of Preparing X-Ray Contrast Compositions Containing Nanoparticles;” U.S. Pat. No. 5,534,270, for “Method of Preparing Stable Drug Nanoparticles;” U.S. Pat. No. 5,510,118, for “Process of Preparing Therapeutic Compositions Containing Nanoparticles;” and U.S. Pat. No. 5,470,583, for “Method of Preparing Nanoparticle Compositions Containing Charged Phospholipids to Reduce Aggregation,” all of which are specifically incorporated by reference.

### Milling to Obtain Nanoparticulate Active Agent Dispersions

According to one aspect of the invention, milling of aqueous active agent dispersions to obtain a dispersion of a nanoparticulate active agent comprises dispersing at least one active agent in a liquid dispersion media in which the active agent is poorly soluble. By “poorly soluble” it is meant that the active agent has a solubility in the liquid dispersion media of less than about 30 mg/ml, less than about 20 mg/ml, preferably less than about 10 mg/ml, and more preferably less than about 1 mg/ml. Such a liquid dispersion media can be, for example, water, aqueous salt solutions, oils such as safflower oil, and solvents such as ethanol, t-butanol, hexane, and glycol.

This is followed by applying mechanical means in the presence of grinding media to reduce the particle size of the active agent to the desired effective average particle size. The active agent particles can be reduced in size in the presence of at least one surface stabilizer. Alternatively, the active agent particles may be contacted with one or more surface stabilizers after attrition. Other compounds, such as a diluent, can be added to the active agent/surface stabilizer composition during the size reduction process. Dispersions can be manufactured continuously or in a batch mode. The resultant nanoparticulate active agent dispersion can then be formulated into a solid form, followed by gamma radiation of the solid form.

### Precipitation to Obtain Nanoparticulate Active Agent Compositions

According to another aspect of the invention, another method of forming the desired nanoparticulate active agent composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the poorly soluble active agent in a suitable solvent; (2) adding the formulation from step (1) to a solution comprising at least one surface stabilizer to form a solution; and (3) precipitating the formulation from step (2) using an appropriate non-solvent. The method can be followed by removal of any formed salt, if present, by dialysis or diafiltration and concentration of the dispersion by conventional means. The resultant nanoparticulate active agent dispersion can then be formulated into a solid form, followed by gamma radiation of the solid form.

### Homogenization to Obtain Nanoparticulate Active Agent Compositions

Exemplary homogenization methods of preparing nanoparticulate active agent compositions are described in U.S. Pat. No. 5,510,118, for “Process of Preparing Therapeutic Compositions Containing Nanoparticles.”

According to another aspect of the invention, such a method comprises dispersing active agent particles in a liquid dispersion medium, followed by subjecting the dispersion to homogenization to reduce the particle size of the active agent to the desired effective average particle size. The active agent particles can be reduced in size in the presence of at least one surface stabilizer. Alternatively, the active agent particles can be contacted with one or more surface stabilizers either before or after particle size reduction. It is preferred, however, to disperse the active agent particles in the liquid dispersion medium in the presence of at least one surface stabilizer as an aid to wetting of the active agent particles. Other compounds, such as a diluent, can be added to the active agent/surface stabilizer composition either before, during, or after the particle size reduction process. Dispersions can be manufactured continuously or in a batch mode. The resultant nanoparticulate active agent dispersion can then be formulated into a solid form, followed by gamma radiation of the solid form.

### Methods of Making Solid Forms of Nanoparticulate Active Agent Compositions

### Spray Drying of Nanoparticulate Active Agent Dispersions

According to an aspect of the invention, solid forms of nanoparticulate active agent dispersions can be prepared by drying the liquid nanoparticulate active agent dispersion following particle size reduction. A preferred drying method is spray drying.

In an exemplary spray drying process, the nanoparticulate active agent dispersion is fed to an atomizer using a peristaltic pump and atomized into a fine spray of droplets. The spray is contacted with hot air in the drying chamber resulting in the evaporation of moisture from the droplets. The resulting spray is passed into a cyclone where the powder is separated and collected. The nanoparticulate active agent dispersion can be spray-dried in the presence or absence of excipients.

The spray-dried powder can be gamma radiated, or the powder can be further processed into a solid dosage form such as a tablet, sachet, etc., followed by gamma radiation of the solid dosage form. Gamma radiated spray-dried powders of nanoparticulate active agents can also be formulated into an aerosol for nasal or pulmonary administration, or the powder can be redispersed in a liquid dispersion media and the subsequent liquid dosage form can be used in a suitable application, such as in oral compositions, injectable compositions, ocular compositions, liquid nasal and pulmonary aerosols, ear drops, etc.

### Lyophilization of Nanoparticulate Active Agent Dispersions

According to an embodiment of the invention, solid or powder forms of nanoparticulate active agent dispersions can also be prepared by lyophilizing the liquid nanoparticulate active agent dispersion following particle size reduction.

In the lyophilization step, water is removed from the nanoparticulate active agent formulations after the dispersion is frozen and placed under vacuum, allowing the ice to change directly from solid to vapor without passing through a liquid phase. The lyophilization process consists of four interdependent processes: freezing, sublimation, the primary drying step, and desorption, which is the secondary drying step. Many lyophilizers can be used to achieve the lyophilization step of nanoparticulate active agent dispersions.

Suitable lyophilization conditions include, for example, those described in EP 0,363,365 (McNeil-PPC Inc.), U.S. Pat. No. 4,178,695 (A. Erbeia), and U.S. Pat. No. 5,384,124 (Farmalyoc), all of which are incorporated herein by reference. Typically, the nanoparticulate active agent dispersion is placed in a suitable vessel and frozen to a temperature of between about −5° C. to about −100° C. The frozen dispersion is then subjected to reduced pressure for a period of up to about 7 days. The combination of parameters such as temperature, pressure, dispersion media, and batch size will impact the time required for the lyophilization process. Under conditions of reduced temperature and pressure, the frozen solvent is removed by sublimation yielding a solid, porous, immediate release solid dosage form having the nanoparticulate active agent distributed throughout.

Following gamma radiation, the lyophilized solid form can be formulated, for example, into a powder, tablet, suppository, or other solid dosage form, a powder can be formulated into an aerosol for nasal or pulmonary administration, or a powder can be reconstituted into a liquid dosage form, such as ocular drops, liquid nasal and pulmonary aerosols, ear drops, injectable compositions, etc.

One embodiment of the invention comprises a method for making a sterilized nanoparticulate docetaxel composition comprising the steps of: mixing docetaxel, optionally including at least one excipient, and at least one surface stabilizer in an aqueous medium containing milling media for a period of time and under conditions sufficient to provide a dispersion of particles of docetaxel having an effective average particle size of less than about 2000 nm and the at least one surface stabilizer adsorbed on the surface of the particles; removing the milling media from the dispersion; lyophilizing the dispersion to form a lyo; and sterilizing the lyo to produce a sterilized docetaxel composition.

### Granulation of Nanoparticulate Active Agent Dispersions

According to a aspect of the invention, a solid form of the invention can be prepared by granulating in a fluidized bed an admixture comprising a nanoparticulate active agent dispersion, comprising at least one surface stabilizer, optionally with a solution of at least one pharmaceutically acceptable water-soluble or water-dispersible excipient, to form a granulate. This can be followed by gamma radiation of the granulate, or gamma radiation of a solid dosage form prepared from the granulate.

### Gamma Radiation

According to an embodiment of the invention, the solid nanoparticulate active agent particles are subjected to gamma radiation at ambient temperature, which remains relatively constant during the period of radiation. Gamma radiation is applied in an amount sufficient to expose the pharmaceutical product to at least 25 kGray of radiation. The total amount of gamma radiation that the solid nanoparticulate active agent is exposed to has been experimentally verified to: (1) render the active agent composition sterile, and (2) maintain the integrity of the nanoparticulate active agent composition. The application of the gamma radiation does not significantly degrade the active agent or reduce the active agent's efficacy. In this way, it is possible to provide products which meet cGMP requirements for sterile products without harming the active agent.

In a preferred aspect of the invention, the gamma radiation is applied in a preferred cumulative amount of about 5 kGray to about 50 kGray or less. Generally, the gamma radiation will normally be applied in a range of about 25 kGray to about 40 kGray or more to provide preferred total dose exposure of about 25 kGray.

One aspect of the invention is that upon reconstitution or redispersion after gamma radiation, the terminally sterilized solid nanoparticulate active agent maintains its overall stability. Specifically the terminally sterilized solid nanoparticulate active agent maintains its redispersibility as evidenced by a retention of particle size, pH, osmolality, assay, and stabilizer concentration following redispersion of the solid in a liquid media.

### Administration of Compositions

In certain embodiments, the present invention provides a method of treating a subject requiring administration of a sterile dosage form. As used herein, the term “subject” is used to mean an animal, preferably a mammal, including a human. The terms “patient” and “subject” may be used interchangeably.

Non-limiting examples of particularly useful applications of such dosage forms include injectable dosage forms, aerosol dosage forms, and dosage forms to be administered to immunocompromised subjects, subjects being treated with immunosuppressants, such as transplant subjects, elderly subjects, and juvenile or infant subjects.

In certain aspects, the sterile dosage forms of the invention can be administered to a subject via any conventional method including, but not limited to, orally, rectally, vaginally, ocularly, parenterally (including, but not limited to, intravenous, intramuscular, or subcutaneous administration), intracisternally, pulmonary, intravaginally, intraperitoneally, locally (including, but not limited to, ointments or drops), via the ear, or as a buccal or nasal spray.

Sterile dosage forms suitable for parenteral injection may include, without limitation, physiologically acceptable sterile aqueous or nonaqueous solutions, dispersions, suspensions or emulsions, and sterile powders for reconstitution into sterile injectable solutions or dispersions. Proper fluidity can be maintained, for example, by the use of a coating such as lecithin, by the maintenance of the required particle size in the case of dispersions, and by the use of surfactants.

Sterile dosage forms for oral administration may include, without limitation, pharmaceutically acceptable emulsions, solutions, suspensions, syrups, and elixirs. In addition to the active agent and surface stabilizer, the sterile dosage forms may include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propylene glycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

In general, the sterile dosage forms according to aspects of the invention will be administered to a mammalian subject in need thereof using a level of drug or active agent that is sufficient to provide the desired physiological effect. The effective amounts of the active agent of the composition of the invention can be determined empirically and can be employed in pure form or, where such forms exist, in pharmaceutically acceptable salt, ester, or prodrug form. Actual dosage levels of the active agent in the sterile dosage form of the invention may be varied to obtain an amount of the active agent that is effective to obtain a desired therapeutic response for a particular composition and method of administration and the condition to be treated. The selected dosage level therefore depends upon the desired therapeutic effect, the route of administration, the potency of the administered active agent, the desired duration of treatment, and other factors. The level of active agent needed to give the desired physiological result is readily determined by one of ordinary skill in the art by referring to standard texts, such as Goodman and Gillman and the Physician's Desk Reference.

Dosage unit compositions may contain such amounts of such submultiples thereof as may be used to make up the daily dose. It will be understood, however, that the specific dose level for any particular subject will depend upon a variety of factors: the type and degree of the cellular or physiological response to be achieved; activity of the specific agent or composition employed; the specific agent(s) or composition employed; the age, body weight, general health, sex, and diet of the patient; the time of administration, route of administration, and rate of excretion of the active agent; the duration of the treatment; active agents used in combination or coincidental with the specific active agent; and like factors well known in the medical arts.

### Method of Treatment

In human therapy, it is important to provide a docetaxel or analogue thereof dosage form that delivers the required therapeutic amount of the drug in vivo, and that renders the drug bioavailable in a constant manner. Thus, another aspect of the present invention provides a method of treating a mammal, including a human, requiring anti-cancer treatment including anti-tumor and anti-leukemia treatment comprising administering to the mammal the nanoparticulate docetaxel or analogue thereof formulation of the invention.

Exemplary types of cancer that can be treated with the nanoparticulate docetaxel or analogue thereof compositions of invention include, but are not limited to, breast, lung (including but not limited to non small cell lung cancer), ovarian, prostate, solid tumors (including but not limited to head and neck, breast, lung, gastrointestinal, genitourinary, melanoma, and sarcoma), primary CNS neoplasms, multiple myeloma, Non-Hodgkin's lymphoma, anaplastic astrocytoma, anaplastic meningioma, anaplastic oligodendroglioma, brain malignant hemangiopericytoma, squamous cell carcinoma of the hypopharynx, squamous cell carcinoma of the larynx, leukemia, squamous cell carcinoma of the lip and oral cavity, squamous cell carcinoma of the nasopharynx, squamous cell carcinoma of the oropharynx, cervical cancer, and pancreatic cancer.

In one embodiment of the invention, the effective dosage for the nanoparticulate docetaxel or analogue thereof compositions of the invention is greater than that required for the comparable non-nanoparticulate docetaxel formulation, e.g., TAXOTERE®. The dosage schedule for TAXOTERE® (docetaxel), which is available in 20 mg (0.5 mL) and 80 mg (2.0 mL) vials, varies with the type of cancer targeted for treatment. For breast cancer, the recommended dosage is 60-100 mg/m2 intravenously over 1 hour every 3 weeks. In cases of non-small cell lung cancer, TAXOTERE® is used only after failure of prior platinum-based chemotherapy. The recommended dosage in this instance is 75 mg/m2 intravenously over 1 hour every 3 weeks. Toxic adverse reactions were reported in patients taking 150 mg/m2 and 200 mg/m2 of TAXOTERE®. In contradistinction, according to one embodiment of the invention, a greater tolerated dosage amount of a docetaxel composition of the present invention may be administered to a patient compared to TAXOTERE® without triggering toxic adverse reactions. The tolerated dosage amount for the present invention may include dosage amounts that are 1%, 5%, 10%, 50%, 100%, 200%, 300%, 400%, 500%, 600%, or 666% greater than the maximum tolerated dose amount reported for TAXOTERE® with no adverse toxic effects, namely, less than 150 mg/m2. Such greater tolerated dosage amounts of the nanoparticulate docetaxel or analogue thereof compositions of the present invention includes dosage amounts greater than about 100, 200, 300, 400, 500, 600, 700, 800, 900 up to 1000 mg/m2.

In another embodiment of the invention, a) aTmax of the docetaxel composition, when assayed in the plasma of a mammalian subject following administration, is less than a Tmax for a non-nanoparticulate docetaxel formulation, administered at the same dosage; (b) a Cmax of the docetaxel composition, when assayed in the plasma of a mammalian subject following administration, is greater than a Cmax for a non-nanoparticulate docetaxel formulation, administered at the same dosage; (c) the AUC of the docetaxel composition, when assayed in the plasma of a mammalian subject following administration, is greater than an AUC for a non-nanoparticulate docetaxel formulation, administered at the same dosage; or (d) any combination thereof.

According to an embodiment of the invention, (a) the Tmax is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tmax exhibited by a non-nanoparticulate docetaxel formulation, administered at the same dosage; (b) the Cmax is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cmax exhibited by a non-nanoparticulate formulation of docetaxel administered at the same dosage; (c) the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of docetaxel administered at the same dosage; or (d) any combination thereof.

According to another aspect of the invention, the composition exhibits a Tmax s of less than about 6 hours, less than about 5 hours, less than about 4 hours, less than about 3 hours, less than about 2 hours, less than about 1 hour, or less than about 30 minutes after administration to fasting subjects.

In one embodiment of the invention, the nanoparticulate docetaxel or analogue thereof composition, including an injectable composition, is free of polysorbate, ethanol, or a combination thereof. In addition, when formulated into an injectable formulation, the compositions of the invention may provide a high concentration in a small volume to be injected. Injectable docetaxel or analogue thereof compositions of the invention can be administered, for example, in a bolus injection or with a slow infusion over a suitable period of time.

One of ordinary skill will appreciate that effective amounts of a docetaxel or analogue thereof can be determined empirically and can be employed in pure form or, where such forms exist, in pharmaceutically acceptable salt, ester, or prodrug form. Actual dosage levels of docetaxel or analogue thereof in the injectable and oral compositions of the invention may be varied to obtain an amount of docetaxel or analogue thereof that is effective to obtain a desired therapeutic response for a particular composition and method of administration. The selected dosage level therefore depends upon the desired therapeutic effect, the route of administration, the potency of the administered docetaxel or analogue thereof, the desired duration of treatment, and other factors.

### EXAMPLES

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

Examples have been set forth below for purposes of illustration and to describe the best mode of the invention at the present time. The scope of the invention is not to be in any way limited by the examples set forth herein.

### Example 1

This example describes two compositions comprising nanoparticulate docetaxel that are chemically and physically stable to sterilizing doses of gamma radiation (at least 25 kGy).

Formulations comprising 10% docetaxel trihydrate, 2.5% povidone K17, 0.5% sodium deoxycholate, and 10% mannitol (all w/w %) in water were processed in a NanoMill-01 equipped with a 100 mL chamber and charged with 500 μm highly crosslinked polystyrene milling media (PolyMill-500). The dispersions were milled for 75-85 minutes at 2930 rpm. Upon completion of milling the particles in one representative experiment had a mean diameter (volume statistics) of 163 nm with D50=159 nm, D90=211 nm, and D95=228 nm. The harvested material from two experiments were combined for use as described below.

A portion of the combined 10% docetaxel dispersion was diluted 1:1 with a 30% sucrose solution to yield a formulation comprising 5% docetaxel, 1.25% povidone K17, 0.25% sodium deoxycholate, 15% sucrose, and 5% mannitol (Formulation 1). A second portion of the 10% dispersion was diluted 1:1 with a 20% sucrose, 10% mannitol solution to yield a formulation comprising 5% docetaxel, 1.25% povidone K17, 0.25% sodium deoxycholate, 10% sucrose, and 10% mannitol (Formulation 2). Samples of both Formulation 1 and Formulation 2 were filled into vials and lyophilized. The final dry composition of Formulation 1 was 18.87% docetaxel, 4.72% povidone K17, 0.94% sodium deoxycholate, 56.60% sucrose, and 18.87% mannitol, and the final dry composition of Formulation 2 was 18.87% docetaxel, 4.72% povidone K17, 0.94% sodium deoxycholate, 37.74% sucrose, and 37.74% mannitol. Vials containing the lyophilized powders were subjected to a range of gamma radiation doses (15, 20, 25, 30, 35, and 40 kGy) and then evaluated for chemical stability and particle size distribution upon reconstitution of water. Formulation 1 was reconstituted with 73.5% water for injection, which resulted in the following concentration of the injectable form of Formulation 1: 5% docetaxel, 1.25% PVP, 0.25% sodium deoxycholate, 15% sucrose, and 5% mannitol. Formulation 2 was reconstituted with 73.5% water for injection, and resulted in the following concentration of the injectable form of Formulation 1: 5% docetaxel, 1.25% PVP, 0.25% sodium deoxycholate, 10% sucrose, and 10% mannitol. The results (Tables 1-4) of the post-sterilized, reconstituted dispersions show that there was no appreciable increase in the average particle size of the docetaxel nanoparticles in either formulation as a result of gamma radiation, nor was there an observable increase in formulation viscosities. Furthermore, chemical analysis indicated that there was only a very modest increase in the impurity profiles of the products.

TABLE 1
Particle Size Data (nm) for Formulation 1 after gamma
radiation and reconstitution

| | Gamma Dose | | | | | | |
|---|---|---|---|---|---|---|---|
| | | | | | | | 40 |
| | 0 kGy | 15 kGy | 20 kGy | 25 kGy | 30 kGy | 35 kGy | kGy |
| Dmean | 172 | 170 | 170 | 169 | 170 | 171 | 171 |
| D50 | 167 | 165 | 165 | 165 | 165 | 166 | 166 |
| D90 | 224 | 222 | 221 | 221 | 222 | 223 | 223 |
| D95 | 247 | 244 | 243 | 243 | 244 | 245 | 245 |

TABLE 2
Potency and Related Substances Data for Formulation 1
after gamma radiation and reconstitution¹

| | Gamma Dose | | | | | | |
|---|---|---|---|---|---|---|---|
| | 0 kGy | 15 kGy | 20 kGy | 25 kGy | 30 kGy | 35 kGy | 40 kGy |
| % Label Claim | 100.5 | 99.7 | 98.6 | 98.8 | 97.7 | 98.5 | 98.8 |
| Total Unknowns | 0.65 | 0.95 | 1.16 | 1.40 | 1.41 | 1.51 | 1.50 |
| % w/w | | | | | | | |

TABLE 3
Particle Size Data (nm) for Formulation 2 after gamma
radiation and reconstitution

| | Gamma Dose | | | | | | |
|---|---|---|---|---|---|---|---|
| | | | | | | | 40 |
| | 0 kGy | 15 kGy | 20 kGy | 25 kGy | 30 kGy | 35 kGy | kGy |
| Dmean | 174 | 175 | 175 | 174 | 174 | 171 | 167 |
| D50 | 169 | 169 | 169 | 168 | 168 | 166 | 163 |
| D90 | 227 | 228 | 228 | 227 | 227 | 223 | 216 |
| D95 | 248 | 250 | 251 | 250 | 250 | 243 | 235 |

TABLE 4
Potency and Related Substances Data for Formulation 2
after gamma radiation and reconstitution¹

| | Gamma Dose | | | | | | |
|---|---|---|---|---|---|---|---|
| | 0 kGy | 15 kGy | 20 kGy | 25 kGy | 30 kGy | 35 kGy | 40 kGy |
| % Label Claim | 99.8 | 102.7 | 100.2 | 98.5 | 98.8 | 99.2 | 100.3 |
| Total Unknowns | 0.76 | 0.86 | 0.95 | 1.20 | 1.09 | 1.25 | 1.34 |
| % w/w | | | | | | | |

¹Reporting threshold = 0.05%

The ratio of amount of drug compared to surface stabilizer (given in percentages) based upon the total combined dry weight of the drug and surface stabilizer, not including other excipients for Formulations 1 and 2 is 80%.

### Example 2

A stable liquid colloidal dispersion of docetaxel was prepared by milling the drug substance in an aqueous solution of povidone (K17), sodium deoxycholate, and dextrose. The final formulation of the liquid composition was 5% docetaxel, 1.25% povidone K17, 0.25% sodium deoxycholate, 20% dextrose, and 73.5% water. When this material was subjected to gamma radiation (15, 20, 25, 30, 35, or 40 kGy) the formulation showed a marked increase in viscosity as a function of gamma dose, and the drug particles that were subjected to >15 kGy of radiation were highly aggregated.

TABLE 5
Particle Size Data (nm) for liquid nanoparticulate docetaxel
formulation after gamma radiation

| | Gamma Dose | | | | | | |
|---|---|---|---|---|---|---|---|
| | 0 kGy | 15 kGy | 20 kGy | 25 kGy | 30 kGy | 35 kGy | 40 kGy |
| Dmean | 161 | 159 | 35,853 | 1,666 | 10,589 | 4,971 | 44,279 |
| D50 | 158 | 156 | 203 | 196 | 247 | 216 | 1,126 |
| D90 | 206 | 202 | 106,704 | 6,537 | 16,415 | 10,878 | 164,559 |
| D95 | 223 | 220 | 132,251 | 10,497 | 64,832 | 15,662 | 195,887 |

This example demonstrates that not every nanoparticulate docetaxel formulation can be sterilized by gamma radiation.

### Example 3

### Formulations 3 and 4

Stable liquid colloidal dispersion of docetaxel was prepared consistent with Examples 1 and 2. The final dry composition of Formulation 3 comprised 18.78% docetaxel, 4.70% povidone K17, 1.39% sodium deoxycholate, 56.35% sucrose, and 18.78% mannitol, and the final dry composition of Formulation 4 was 18.78% docetaxel, 4.70% povidone K17, 1.39% sodium deoxycholate, 37.57% sucrose, and 37.57% mannitol. Vials containing the lyophilized powders were subjected to a range of gamma radiation doses (15, 20, 25, 30, 35, and 40 kGy) and then evaluated for chemical stability and particle size distribution upon reconstitution of water. Formulation 3 was reconstituted with 73.38% water for injection, which resulted in the following concentration of the injectable form of Formulation 3: 5% docetaxel, 1.25% PVP, 0.37% sodium deoxycholate, 15% sucrose, and 5% mannitol. Formulation 4 was reconstituted with 73.38% water for injection, and resulted in the following concentration of the injectable form of Formulation 4: 5% docetaxel, 1.25% PVP, 0.37% sodium deoxycholate, 10% sucrose, and 10% mannitol.

All numerical ranges described herein include all combinations and subcombinations of ranges and specific integers encompassed therein.

Various modifications of the invention, in addition to those described herein, will be apparent to those skilled in the art from the foregoing description. Such modifications are also intended to fall within the scope of the appended claims.

What is claimed is:

1. A sterile composition comprising:
(a) particles comprising at least one active agent selected from the group consisting of docetaxel, salts of docetaxel, derivatives of docetaxel, conjugates of docetaxel and analogues of docetaxel, wherein the particles have an effective average particle size of less than about 2000 nm; and
(b) at least one surface stabilizer adsorbed on a surface of the particles, wherein the composition is sterilized by exposure to gamma radiation.

2. The composition of claim 1, wherein the active agent is in a form selected from the group consisting of crystalline, amorphous, semi-crystalline, semi-amorphous, and mixtures thereof.

3. The composition of claim 1, wherein the active agent is docetaxel.

4. The composition of claim 3, wherein the docetaxel is in a form selected from the group consisting of an anhydrous, a hydrated, and a triydrate crystal form, and mixtures thereof.

5. The composition of claim 1, wherein the effective average particle size is selected from the group consisting of less than: about 1900 nm, about 1800 nm, about 1700 nm, about 1600 nm, about 1500 nm, about 1400 nm, about 1300 nm, about 1200 nm, about 1100 nm, about 1000 nm, about 900 nm, about 800 nm, about 700 nm, about 650 nm, about 600 nm, about 550 nm, about 500 nm, about 450 nm, about 400 nm, about 350 nm, about 300 nm, about 250 nm, about 200 nm, about 150 nm, about 100 nm, about 75 nm, and about 50 nm.

6. The composition of claim 1, wherein the composition is formulated:
(a) for routes of administration selected from the group consisting of oral, pulmonary, rectal, opthalmic, colonic, parenteral, intracisternal, intravaginal, intraperitoneal, local, buccal, nasal, and topical administration;
(b) into a dosage form selected from the group consisting of liquid dispersions, solid dispersions, liquid-filled capsules, gels, aerosols, ointments, creams, lyophilized formulations, tablets, capsules, multi-particulate filled capsules, tablets composed of multi-particulates, compressed tablets, and capsules filled with enteric-coated beads of the active agent;
(c) into a dosage form selected from the group consisting of controlled release formulations, fast melt formulations, delayed release formulations, extended release formulations, pulsatile release formulations, and mixed immediate release and controlled release formulations; or
(d) any combination of (a), (b), and (c).

7. The composition of claim 6, wherein the composition is an injectable formulation.

8. The composition of claim 6, wherein the composition is formulated for pulmonary administration.

9. The composition of claim 6, wherein the composition is in a solid form.

10. The composition of claim 6, wherein the composition is in a liquid form.

11. The composition of claim 1, wherein:
(a) the at least one surface stabilizer is present in an amount selected from the group consisting of about 1%, 5%, 10%, 20%, 30%, 40%, 50%, 60%, 70%, 80%, 90%, 95% or 99%, by weight, based on the total combined dry weight of the active agent and the at least one surface stabilizer, not including other excipients;
(b) the particles are present in an amount selected from the group consisting of about 1%, 5%, 10%, 20%, 30%, 40%, 50%, 60%, 70%, 80%, 90%, 95% or 99%, by weight, based on the total combined weight of the particles comprising the active agent and the at least one surface stabilizer, not including other excipients; or
(c) a combination of (a) and (b).

12. The composition of claim 1, wherein the at least one surface stabilizer is selected from the group consisting of an anionic surface stabilizer, a cationic surface stabilizer, a zwitterionic surface stabilizer, a non-ionic surface stabilizer, and an ionic surface stabilizer.

13. The composition of claim 1, wherein the at least one surface stabilizer is selected from the group consisting of povidone, cetyl pyridinium chloride, albumin, human serum albumin, bovine serum albumin, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, sodium deoxycholate, alkyl aryl polyether sulfonates, mixtures of sucrose stearate and sucrose distearate, p-isononylphenoxypoly-(glycidol), decanoyl-N-methylglucamide; n-decyl b-D-glucopyranoside; n-decyl b-D-maltopyranoside; n-dodecyl b-D-glucopyranoside; n-dodecyl b-D-maltoside; heptanoyl-N-methylglucamide; n-heptyl-b-D-glucopyranoside; n-heptyl b-D-thioglucoside; n-hexyl b-D-glucopyranoside; nonanoyl-N-methylglucamide; n-noyl b-D-glucopyranoside; octanoyl-N-methylglucamide; n-octyl-b-D-glucopyranoside; octyl b-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)4 ammonium chloride, lauryl dimethyl(ethenoxy)4 ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

14. The composition of claim 13, wherein the at least one surface stabilizer is povidone.

15. The composition of claim 13, wherein the at least one surface stabilizer is sodium deoxycholate.

16. The composition of claim 1, wherein the at least one surface stabilizer is selected from the group consisting of poloxamer 188, poloxamer 338, poloxamer 407, polysorbate 80, and lecithin.

17. The composition of claim 1 further comprising at least one excipient.

18. The composition of claim 1, wherein the at least one surface stabilizer is a protein.

19. The composition of claim 18, wherein the surface stabilizer is an albumin.

20. The composition of claim 19, wherein the albumin is human serum albumin.

21. The composition of claim 17 wherein the at least one excipient is a sugar selected from the group consisting of sucrose, mannitol, dextrose, lactose, sorbitol, maltose, and trehalose.

22. The composition of claim 17, wherein the at least one excipient is selected from the group consisting of a bulking agent, a crystal growth inhibitor, a free radial scavenger agent, and a redispersion agent.

23. The composition of claim 17, wherein the at least one excipient is present in the amount selected from the group consisting of from about 5 to about 95, about 10 to about 95, about 20 to about 95, about 50 to about 90, about 60 to about 90, about 70 to about 90, or about 70 to about 80, measured by % w/w of the dry composition.

24. The composition of claim 1, wherein the gamma radiation provides a total dose of radiation selected from the group consisting of from about 5 to about 50 kGray, about 15 kGray to about 40 kGray, about 15 to about 30 kGray, and about 20 to about 30 kGray.

25. The composition of claim 1, wherein the gamma radiation provides a total dose of about 25 kGray.

26. A dry composition comprising about 18.87% docetaxel, about 4.72% povidone, about 0.94% sodium deoxycholate, about 56.60% sucrose, and about 18.87% mannitol.

27. A dry composition comprising about 18.87% docetaxel, about 4.72% povidone, about 0.94% sodium deoxycholate, about 37.74% sucrose, and about 37.74% mannitol.

28. The composition of claim 1, wherein the active agent is selected from the group consisting of:
(a) docetaxel analogues comprising cyclohexyl groups instead of phenyl groups at the C-3′ benzoate position, the C-2 benzoate positions, or a combination thereof;
(b) docetaxel analogues lacking phenyl or an aromatic group at C-3′ or C-2 position;
(c) 2-amido docetaxel analogues;
(d) docetaxel analogues lacking the oxetane D-ring but possessing the 4alpha-acetoxy group;
(e) 5(20)deoxydocetaxel;
(f) 10-deoxy-10-C-morpholinoethyl docetaxel analogues;
(g) analogues having a t-butyl carbamate as the isoserine N-acyl substituent, but differing from docetaxel at C-10 (acetyl group versus hydroxyl) and at the C-13 isoserine linkage (enol ester versus ester);
(h) docetaxel analogues having a peptide side chain at C3;
(i) XRP9881 (10-deacetyl baccatin III docetaxel analogue);
(j) XRP6528 (10-deacetyl baccatin III docetaxel analogue);
(k) Ortataxel (14-beta-hydroxy-deacetyl baccatin III docetaxel analogue);
(l) MAC-321 (10-deacetyl-7-propanoyl baccatin docetaxel analogue);
(m) DJ-927 (7-deoxy-9-beta-dihydro-9,10, 0-acetal taxane docetaxal analogue);
(n) docetaxel analogues having C2-C3′N-linkages bearing an aromatic ring at position C2, and tethered between N3′ and the C2-aromatic ring at the ortho position;
(o) docetaxel analogues having C2-C3′N-linkages bearing an aromatic ring at position C2, and tethered between N3′ and the C2-aromatic ring at the meta position;
(p) docetaxel analogues bearing 22-membered (or more) rings connecting the C-2OH and C-3′ NH moieties;
(q) 7beta-O-glycosylated docetaxel analogues;
(r) 10-alkylated docetaxel analogues;
(s) 2′,2′-difluoro docetaxel analogues;
(t) 3′-(2-furyl) docetaxel analogues;
(u) 3′-(2-pyrrolyl) docetaxel analogues; and
(v) fluorescent and biotinylated docetaxel analogues.

29. The composition of claim 28, wherein the docetaxel analogue is selected from the group consisting of:
(a) 3′-dephenyl-3′cyclohexyldocetaxel;
(b) 2-(hexahydro)docetaxel;
(c) 3′-dephenyl-3′cyclohexyl-2-(hexahydro)docetaxel;
(d) 3′-dephenyl-3′-cyclohexyldocetaxel;
(e) 2-(hexahydro)docetaxel;
(f) m-methoxy docetaxel analogues;
(g) m-chlorobenzoylamido docetaxel analogues;
(h) 5(20)-thia docetaxel analogues;
(i) docetaxel analogues in which the 7-hydroxyl group is modified to the hydrophobic group methoxy;
(j) docetaxel analogues in which the 7-hydroxyl group is modified to the hydrophobic group deoxy;
(k) docetaxel analogues in which the 7-hydroxyl group is modified to the hydrophobic group 6,7-olefin;
(l) docetaxel analogues in which the 7-hydroxyl group is modified to the hydrophobic group alpha-F;
(m) docetaxel analogues in which the 7-hydroxyl group is modified to the hydrophobic group 7-beta-8-beta-methano;
(n) docetaxel analogues in which the 7-hydroxyl group is modified to the hydrophobic group fluoromethoxy;
(o) 10-alkylated docetaxel analogue having a methoxycarbonyl group at the end of the alkyl moiety;
(p) docetaxel analogues that possess a N-(7-nitrobenz-2-oxa-1,3-diazo-4-yl)amido-6-caproyl chain in position 7 or 3′;
(q) docetaxel analogues that possess a N-(7-nitrobenz-2-oxa-1,3-diazo-4-yl)amido-3-propanoyl group at 3′; and
(r) docetaxel analogues that possess a 5′-biotinyl amido-6-caproyl chain in position 7, 10 or 3′.

30. A method for making a sterilized nanoparticulate composition comprising the steps of:
lyophilizing an aqueous dispersion comprising at least one active agent selected from the group consisting of docetaxel, salts of docetaxel, derivatives of docetaxel, conjugates of docetaxel and analogues of docetaxel, wherein the particles have an effective average particle size of less than about 2000 nm, and at least one surface stabilizer adsorbed on a surface of the particles, to form a lyo; and
sterilizing the lyo to produce a sterilized composition.

31. The method of claim 30, further comprising before the lyophilizing step, the step of mixing the at least one active agent selected from the group consisting of docetaxel, salts of docetaxel, derivatives of docetaxel, conjugates of docetaxel and analogues of docetaxel, and the at least one surface stabilizer in an aqueous medium for a period of time and under conditions sufficient to provide the aqueous dispersion.

32. The method of claim 31, wherein the mixing step is selected from the group consisting of milling, attrition, homogenizing, precipitating, supercritical fluids processing, freezing, nano-electrospraying techniques, or any combination thereof.

33. The method of claim 30, wherein the sterilizing step comprises exposing the lyo to a gamma radiation dose selected from the group consisting of from about 5 to about 50 kGray, about 15 kGray to about 40 kGray, about 15 to about 30 kGray, and about 20 to about 30 kGray.

34. The method of claim 30, wherein the sterilizing step comprises exposing the lyo to about 25 kGray of gamma radiation.

35. The method of claim 30, wherein the aqueous dispersion further comprises at least one excipient selected from the group consisting of a bulking agent, a crystal growth inhibitor, a free radical scavenger agent, and a redispersion agent.

36. The method of claim 30, wherein the aqueous dispersion before the lyophilizing step has an effective average particle size selected from the group consisting of less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1 micron, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

37. The method of claim 30 further comprising, after the sterilizing step, the step of redispersing the lyo in an aqueous medium forming a post-sterilized dispersion having an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1 micron, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

39. A terminally sterilized lyophilization composition made from the steps comprising:
milling at least one active agent selected from the group consisting of docetaxel, salts of docetaxel, derivatives of docetaxel, conjugates of docetaxel and analogues of docetaxel, an excipient selected from the group consisting of a bulking agent, a crystal growth inhibitor, a free radical scavenger agent, a redispersion agent, and at least one surface stabilizer, with milling media in an aqueous medium for a period of time and under conditions sufficient to provide a dispersion of particles of the at least one active agent having an effective average particle size of less than about 2000 nm, and the at least one surface stabilizer adsorbed on the surface of the particles;
removing the milling media from the dispersion;
lyophilizing the dispersion to form a lyo; and
sterilizing the lyo to produce a sterilized composition.

40. The composition of claim 39, wherein the sterilizing step comprises exposing the lyo to a dose of gamma radiation effective to produce sterilization.

41. A method of treating a subject in need of docetaxel or a salt, derivative, conjugate or analogue thereof comprising administering to the subject an effective amount of a composition comprising:
(a) particles comprising docetaxel, a salt, derivative, conjugate or analogue thereof, wherein the particles have an effective average particle size of less than about 2000 nm; and
(b) at least one surface stabilizer adsorbed on a surface of the particles,
wherein the composition is sterilized by exposure to gamma radiation.

42. The method of claim 41, wherein the composition is administered by injection.

43. A sterile liquid dosage form of docetaxel for intravenous administration comprising:
(a) about 5% by weight particles of at least one active agent selected from the group consisting of docetaxel, salts of docetaxel, derivatives of docetaxel, conjugates of docetaxel and analogues of docetaxel, the particles having an effective average particle size of less than about 2000 nm;
(b) two surface stabilizers, one or both of the surface stabilizers is adsorbed on a surface of particles;
(c) sucrose; and
(d) mannitol,
wherein the composition is sterilized by exposure to gamma radiation, and wherein the composition is administered to a patient at a dosage amount selected from the group consisting of about 100, 200, 300, 400, 500, 600, 700, 800, 900 and 1000 mg/m².
